(19) United States
(12) Patent Application Publication
(10) Pub. No.: US 2017/0340477 A1
(43) Pub. Date: Nov. 30, 2017

(54) Cryogenic Therapy Chamber

(71) Applicants: Brandon Greene, Lexington, KY (US); Crystal Greene, Lexington, KY (US)

(72) Inventors: Brandon Greene, Lexington, KY (US); Crystal Greene, Lexington, KY (US)

(21) Appl. No.: 15/166,381

(22) Filed: May 27, 2016

(51) Int. Cl. A61F 7/00

(52) U.S. Cl. CPC A61F 7/0053

(57) ABSTRACT

A kit for the assembly of a cryogenic chamber, including at least four wall modules including respective bodies of insulation; wherein the at least four wall modules include at least two end wall modules and at least two lateral wall modules; wherein the at least four wall modules include at least one vent wall module including a lumen surrounded by insulation, a connector extending from a surface of the vent wall module to the lumen, and a vent extending from the surface of the vent wall module to the lumen, wherein the connector is in fluid communication with the vent via the lumen; and an air chiller that is connectable to the connector of the vent module such that the air chiller is in fluid communication with the vent.

### FIELD OF THE INVENTION

The present invention relates to cryogenic therapy and more particularly to a method and apparatus for performing cryogenic therapy.

### BACKGROUND OF THE INVENTION

There are known methods in medical technology for performing cryogenic therapy by exposing certain parts of the human body or the whole body to agents that reduce temperature. These methods are generally carried out by using liquefied gases or compressed gases.

However, any previously disclosed cryogenic therapy methods and cryogenic therapy chambers do not include modular kits that allow the assembly of a custom-sized chamber based on the end-user's needs. Thus, there remains a need for modularized cryogenic chamber kits.

### SUMMARY OF THE INVENTION

Embodiments of the present invention relate to a kit for the assembly of a cryogenic chamber, including at least four wall modules including respective bodies of insulation; wherein the at least four wall modules include at least two end wall modules and at least two lateral wall modules; wherein the at least four wall modules include at least one vent wall module including a lumen surrounded by insulation, a connector extending from a surface of the vent wall module to the lumen, and a vent extending from the surface of the vent wall module to the lumen, wherein the connector is in fluid communication with the vent via the lumen; and an air chiller that is connectable to the connector of the vent module such that the air chiller is in fluid communication with the vent.

Further embodiments of the present invention include a vent wall module for a cryogenic chamber, including a lumen surrounded by an insulation, a vent extending from a surface of the vent wall module to the lumen, and a connector extending from the surface of the vent wall module to the lumen, wherein the connector and vent are in fluid communication via the lumen.

It is understood that other embodiments of the present invention will become readily apparent to those skilled in the art from the following detailed description, wherein it is shown and described only various embodiments of the invention by way of illustration. As will be realized, the invention is capable of other and different embodiments and its several details are capable of modification in various other respects, all without departing from the spirit and scope of the present invention. Accordingly, the drawings and detailed description are to be regarded as illustrative in nature and not as restrictive.

### BRIEF DESCRIPTION OF THE DRAWINGS

FIG. 1A depicts a diagram of a top-down view of a cryogenic stall constructed from a cryogenic stall log module kit in accordance with the principles of the present invention.

FIG. 1B depicts a diagram of a lateral view of the cryogenic stall of FIG. 1A looking toward the second lateral wall in accordance with the principles of the present invention.

FIG. 1C depicts a diagram of an end view of the cryogenic stall of FIG. 1A looking toward a first end wall, wherein the first end wall comprises a neck scarflet in accordance with the principles of the present invention.

FIG. 1D depicts a diagram of an end view of the cryogenic stall of FIG. 1D looking toward a second end wall, wherein the second end wall comprises a door in accordance with the principles of the present invention.

FIG. 2 depicts diagrams of individual logs, a vent plug, and a log plug of the kit for building the cryogenic stall of FIG. 1A in accordance with the principles of the present invention.

FIG. 3 depicts a cross-sectional view diagram of a vent log of FIG. 1A in accordance with the principles of the present invention.

FIG. 4A depicts a diagram of an embodiment of the neck scarflet of FIG. 1C in accordance with the principles of the present invention.

FIG. 4B depicts an alternative neck scarflet in accordance with the principles of the present invention.

FIG. 4C depicts another alternative neck scarflet wherein supports may be attached to or integral with a door in accordance with the principles of the present invention.

FIG. 5A depicts a diagram of an embodiment of the door of FIG. 1D in accordance with the principles of the present invention.

FIG. 5B represents an alternative embodiment of a door of the kit in accordance with the principles of the present invention.

FIG. 6A depicts a representation of a cryogenic stall constructed of slab modules in accordance with the principles of the present invention.

FIG. 6B depicts a view of the outside of a lateral slab wall in accordance with the principles of the present invention.

FIG. 6C depicts a view of the inside of the lateral slab wall in accordance with the principles of the present invention.

FIG. 7 represents a roof of the kit in accordance with the principles of the present invention.

FIG. 8 depicts a floor tray of the kit in accordance with the principles of the present invention.

FIG. 9 represents a treadmill of the kit in accordance with the principles of the present invention.

FIG. 10 depicts a cryogenic constructed of brick modules of the present invention.

FIG. 11 is a depiction of the inside of the first lateral wall of the cryogenic stall according to the principles of the present invention.

FIG. 12 is a depiction of the outside of the first end wall of the cryogenic stall according to the principles of the present invention.

FIG. 13 depicts the second end wall of the cryogenic stall according to the principles of the present invention.

FIG. 14 depicts a brick of the kit for the cryogenic stall in accordance with the principles of the present invention.

FIG. 15 depicts an end brick of the kit for constructing the cryogenic stall in accordance with the principles of the present invention.

FIG. 16 depicts a frame of the kit in accordance with the principles of the present invention.

FIG. 17 depicts a vent brick of the kit in accordance with the principles of the present invention.

FIG. 18 illustrates a block-level diagram of a computer in accordance with the principles of the present invention.

### DETAILED DESCRIPTION OF THE PREFERRED EMBODIMENTS

“About,” as used in this application, means within plus or minus one at the last reported digit. For example, about 1.00 means 1.00±0.01 unit.

“Around,” when used to describe a unit or percentage, means within plus or minus one unit or plus or minus one percentage point.

“Substantially,” as used in this application with reference to an angle, means within one degree. For example, substantially planar means within one degree counterclockwise and within one degree clockwise of planar orientation.

“Substantially similar,” as used in this application, means having at least each of the properties of the referenced structure plus the additional structure disclosed. If the additional structure conflicts, the additional structure supersedes the structure incorporated by reference.

For the purposes of this disclosure, “and” and “or” shall be construed as conjunctively or disjunctively, whichever provides the broadest disclosure in each instance of use of “and” and “or.”

For the purposes of this disclosure, structures disclosed in singular form are not limited to a single structure, but can include multiple instances of the disclosed structure, unless specifically stated otherwise.

The dimensions disclosed for one embodiment of the cryogenic stall also apply to the other embodiments for the cryogenic stall. For example, a 183 cm (6 foot) height disclosed for the slab module embodiment also applies such that the log embodiment may contain sufficient logs to build the cryogenic stall to the 183 cm height. This disclosure also applies to the brick embodiments, such that one of ordinary skill would understand that sufficient bricks to build the cryogenic stall the 183 cm height may be included in the kit.

The detailed description set forth below in connection with the appended drawings is intended as a description of various embodiments of the invention and is not intended to represent the only embodiments in which the invention may be practiced. The detailed description includes specific details for the purpose of providing a thorough understanding of the invention. However, it will be apparent to those skilled in the art that the invention may be practiced without these specific details. In some instances, well known structures and components are shown in block diagram form in order to avoid obscuring the concepts of the invention.

Cryogenic chambers can include a device for preparing cryogenically chilled air which can be provided to the interior of the cryogenic chamber. The device can include an air compressor, a dryer for removing the steam from the compressed air, a heat exchanger and a liquefied gas container. The device may also include measuring elements, control elements and protective elements. The parts of the device which contain the cryogenic agents may be provided within a thermal-insulated system.

The cryogenic chamber kit of the present invention may be used to construct a custom-sized cryogenic therapy chamber. The size may be determined by the needs of the end-user. For example, the end-user may be a veterinarian with many dog-owning customers. Thus, the veterinarian may size the cryogenic therapy chamber to fit dogs, such that excess energy is not wasted and that the dog is efficiently chilled without suffocating the dog.

Alternatively, equine veterinarians may use the kit to construct a larger cryogenic therapy chamber sized to efficiently cool horses without suffocating the horses.

The kit of the present invention may include wall modules for building the cryogenic chamber. The wall modules may include a body comprising materials sufficient to retain rigidity of the wall module and may further comprise insulation capable of withstanding cryogenic temperatures. Example techniques and materials include fiberglass, certain minerals, certain plastics, certain polymers, rubber, a sealed vacuum, etc.

Furthermore, the kit of the present invention may be practiced in various embodiments, such as a kit of log modules, a kit of slab modules, and a kit of brick modules. Each of these modules may be used to construct one or more end walls and one or more lateral walls of a cryogenic stall for use with cryogenic therapy. Thus, end wall modules can include one of log modules, slab modules, or brick modules. Additionally, lateral wall modules can include one of log modules, slab modules, or brick modules. In some embodiments, the type of modules for the end wall will correspond to the type of modules for the lateral wall.

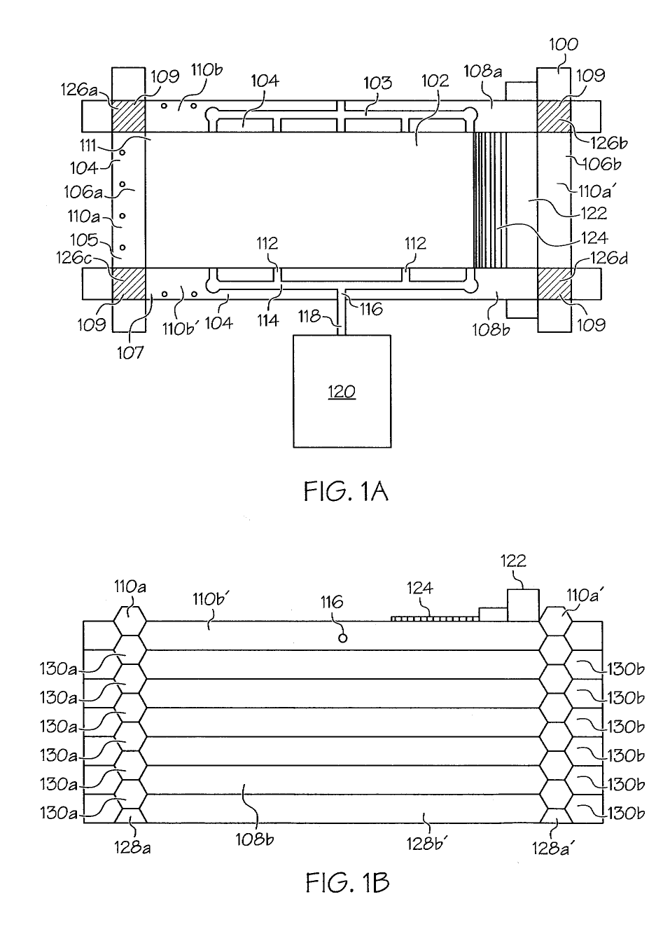

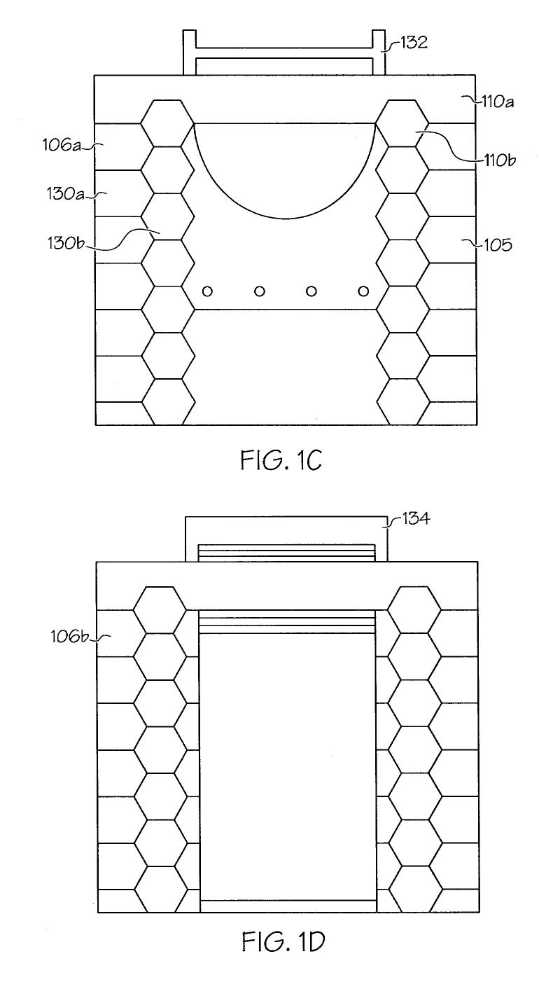

FIG. 1A depicts a diagram of a top-down view of a cryogenic stall 100 constructed from a cryogenic stall log module kit in accordance with the principles of the present invention.

Embodiments of the present invention may include a kit for constructing a cryogenic therapy chamber, such as cryogenic stall 100. This kit may include logs 103, vent plugs 206, log plugs 208, one or more doors 134, a neck scarflet 132, one or more air chillers 120, a hose 118, a floor cover 111, and/or a roof. Logs 103 can include end logs 105, such as end foundation logs 128a, end builder logs 130a, and/or end vent logs 110a. Logs 103 can also include lateral logs 107, such as lateral foundation logs 128b, lateral builder logs 130b, and/or lateral vent logs 110b.

Log module kits may include lateral logs 107 and end logs 105. These logs 103 can be stacked in an interlocking manner such that corresponding walls 104 are erected to form cryogenic stall 100. For example, a first stack of end logs 105 may form a first end wall 106a at a first end of the cryogenic stall 100. A second stack of end logs 105 may form a second end wall 106b at a second end of the cryogenic stall 100.

A first stack of lateral logs 107 may interlock with the corresponding end logs 105 of the first end wall 106a and of the second end wall 106b at respective intersections 126a and 126b. Thus, the first stack of lateral logs 107 may form a first lateral wall 108a spanning from at least first end wall 106a at intersection 126a to second end wall 106b at intersection 126b.

A second stack of lateral logs 107 may interlock with the corresponding end logs 105 of the first end wall 106a and of the second end wall 106b. However, the second stack of lateral logs 107 may interlock with end walls 106a, 106b at respective notches 109 at an opposite end of the corresponding end logs 105 compared to the interlocking intersections 126a, 126b, 126c, 126d of the first lateral wall 108a. Thus, the second stack of lateral logs 107 may form a second lateral wall 108b spanning from at least first end wall 106a at intersection 126c to second end wall 106b at intersection 126d.

Intersections 126a, 126b, 126c, 126d may be formed at the interlocking intersection of each respective interlocking notch 109 of the logs 103. Thus, a recessed plane of each notch 109 of each respective log 103 may rest on the corresponding notch 109 of the interlocking log 103 immediately below. Each interlocking log 103 may interlock at a substantially perpendicular intersections with the interlocking logs 103 immediately above and immediately below each respective log 103. Thus, lateral walls 108a, 108b may be substantially parallel. End walls 106a, 106b may be substantially parallel. Lateral walls 108a, 108b may be substantially perpendicular to end walls 106.

The respective lateral logs 107 may be longer than the corresponding end logs 105. The lateral logs 107 may be stacked to form lengthwise lateral walls 108a, 108b of the cryogenic stall 100. The end logs 105 may be stacked in an interlocking manner with the lateral logs 107 to form end walls 106a, 106b at the ends of each lateral wall 108. The lateral logs 107 may also be stacked in interlocking relationship with a log of the neck scarflet or a log of a door.

A cryogenic chamber 102 may comprise an area within the walls 104 of the cryogenic stall 100. The cryogenic chamber 102 may be sized to receive an animal, such as a horse or a dog. In some embodiments, the cryogenic chamber 102 may be sized to receive a human. The cryogenic chamber 102 may also receive chilled air such that any contained subject animal within the chamber 102 is chilled.

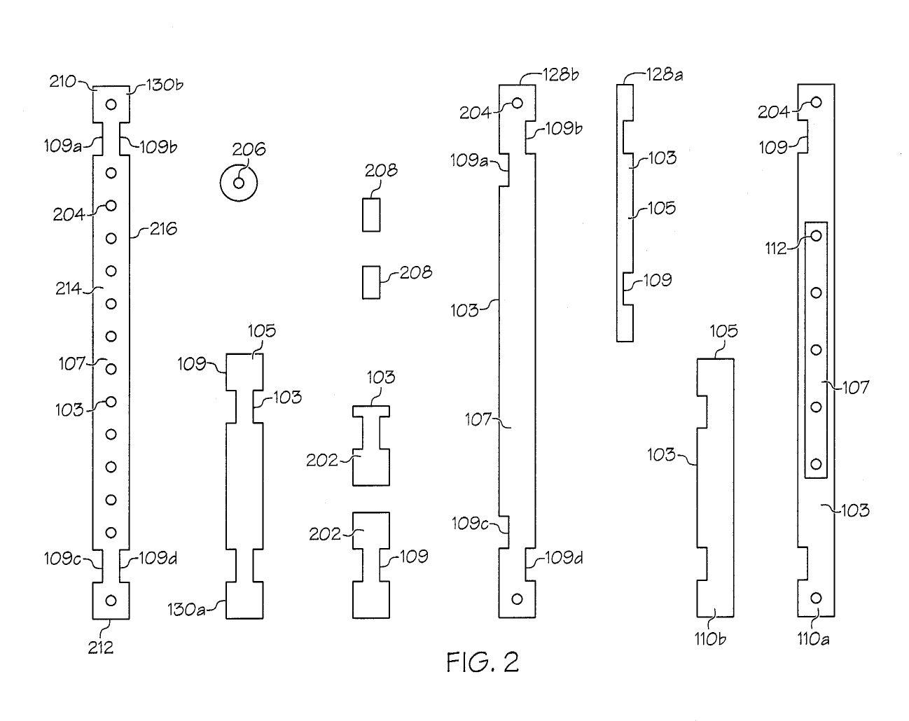

The logs 103 may also include vent logs 110a, 110b and foundation logs 128a, 128b, as depicted in FIG. 2. The logs 103 may be used to construct the cryogenic stall 100 of FIG. 1A. The vent logs 110a, 110b may comprise a structure sufficient for the delivery of chilled air into the cryogenic chamber 102. The vent logs 110a, 110b may include vents 112 and at least one connector 116 for an input hose 118 through which the hose 118 may be in fluid communication with the vent log 110. In some embodiments, only the lateral vent logs 110b contain respective connectors 116. In other embodiments, the kit may be constructed with two air chillers 120 and two hoses 118 connected to both lateral walls 108a, 108b. In some embodiments, only one lateral wall 108a, 108b is connected to the hose 118 and air chiller 120. The builder logs 130a, 130b and foundation logs 128a, 128b may not necessarily comprise such a fluid transport system, and may comprise solid bodies (e.g. integrally formed materials throughout the log 103). The foundation logs 128a, 128b may also comprise solid bodies and may further include a substantially flat level portion opposite a rounded side comprising notches 109. This level portion may be placed directly on the ground and the remaining logs 103 stacked in interlocking fashion onto the foundation logs 103. The vent logs 110a, 110b of the kit may include one or more respective end vent logs 110a and one or more respective lateral vent logs 110b. The foundation logs 103 of the kit may include one or more respective end foundation logs 128a and one or more respective lateral foundation logs 128b.

FIG. 1B depicts a diagram of a side view of the cryogenic stall 100 of FIG. 1A looking toward the second lateral wall 128′ in accordance with the principles of the present invention.

In some embodiments, the corresponding notches 109a, 109b, 109c, 109d of the end vent logs 110a, 110a′ (e.g. the notches 109a, 109b, 109c, 109d on a bottom side of the vent log 110a, 110a′, 110b, 110b′, when the vent log 110a, 110a′, 110b, 110b′ is positioned for building the stall 100) may rest on the corresponding notches 109a, 109b, 109c, 109d of the lateral vent logs 110b, 110b′. The notches 109 of the lateral vent logs 110b may rest on the corresponding notches 109 of the end builder logs 130a. The notches 109 of the end builder logs 130a may rest on the corresponding notches 109 of the lateral builder logs 130b. The end builder logs 130a and lateral builder logs 130b may be stacked in the same manner for one or more layers. The lowest end builder log 130a may be stacked on lateral foundation logs 128b. The lateral foundation logs 128b may be stacked on end foundation logs 128a. Therefore, the corresponding notches 109 of lateral foundation logs 128b may rest upon the corresponding notches 109 of end foundation logs 128a. End foundation logs 128a or lateral foundation logs 128b may rest upon the ground.

Embodiments also include the corresponding notches 109 of the lateral vent logs 110b resting on the end vent logs 110a. The notches 109 of the end vent logs 110a may also rest on the corresponding notches 109 of the lateral builder logs 130b. The corresponding notches 109 of the lateral builder logs 130b may rest on the corresponding notches 109 of the end builder logs 130a. One or more layers of builder logs 130a, 130b may rest upon subsequent layers of builder logs 130a, 130b in a similar interlocking manner. The lowest end builder log 103a or lowest lateral builder log 130b may be stacked on the foundation logs 128a, 128b. The corresponding notches 109 of end builder log 130a or lateral builder log 130b may rest upon the corresponding notches 109 of end foundation log 128a or lateral foundation log 128b. In some embodiments, the end foundation logs 128a may be stacked on lateral foundation logs 128b. Therefore, the corresponding notches 109 of end foundation logs 128a may rest upon the corresponding notches 109 of lateral foundation logs 128b. Lateral foundation logs 128b or end foundation logs 128a may rest upon the ground.

One of ordinary skill realizes that the order of the logs 103 may be mixed, one or more logs 103 may be removed, or otherwise stacked in a different order than described above. Furthermore, the altered stacking may reach the same result as occurs with the immediately described order. For example, vent logs 110a, 110b may be stacked between layers of builder logs 130a, 130b. Alternatively, embodiments include omitting the foundation logs 128a, 128b.

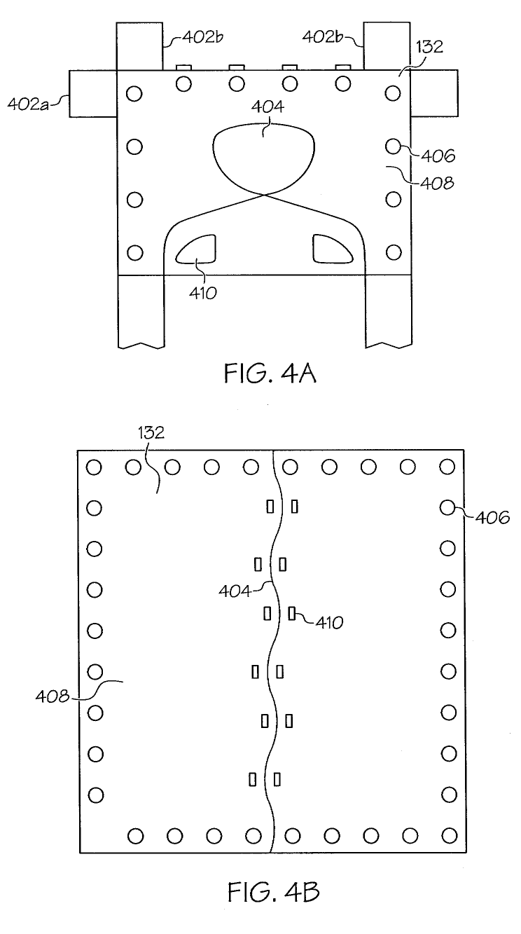

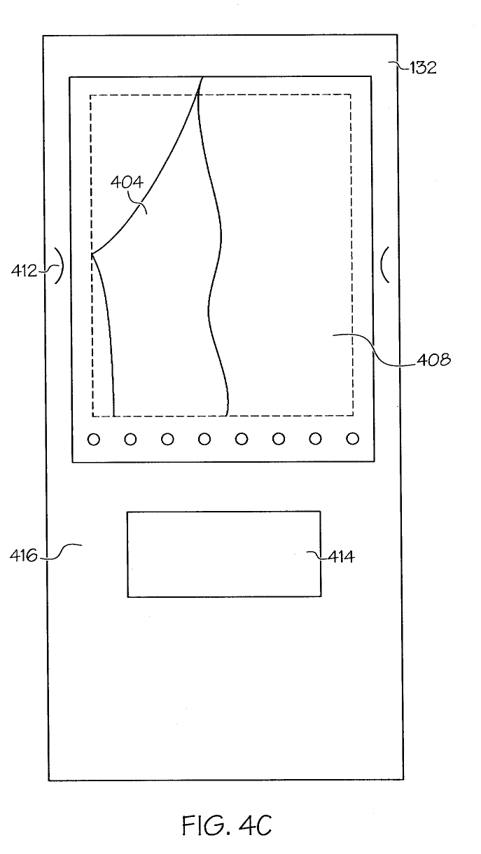

FIG. 1C depicts a diagram of an end view of the cryogenic stall 100 of FIG. 1A looking toward a first end wall 106a, wherein the first end wall 106a comprises a neck scarflet (described with respect to FIGS. 4A and 4B) in accordance with the principles of the present invention. As depicted, the neck scarflet may be integrated with the first end wall 106a. For example, the neck scarflet may include a frame with an attached or integrated log. The log of the neck scarflet may interlock with the lateral logs 107 such that the log of the neck scarflet is stacked into first end wall 106a in vertical alignment with end logs 105. The neck scarflet may be integrated into a swinging door that opens inward or outward. Alternatively, the neck scarflet may be integrated into a tambour door that raises. Furthermore, the frame of the neck scarflet may comprise an interlock mechanism (described further with respect to FIGS. 2, 4 and 5) for attachment to the side of the lateral logs 107. For example, the interlock mechanism may include a male interlock and a female interlock. The male interlock may comprise a rod. The female interlock may include a female interlock formed to receive the male rod. Other examples include any other attachment or stabilization mechanism, such as adhesives, hook and loop, magnets, etc.

In embodiments wherein the scarflet frame does not include sufficient logs to engage with the notches 109 of all the lateral logs 107 beside the scarflet at the first end wall 106a, stub logs (described with respect to FIG. 2) may be used to engage the corresponding notches 109 of the lateral logs 107 above and below the gaps.

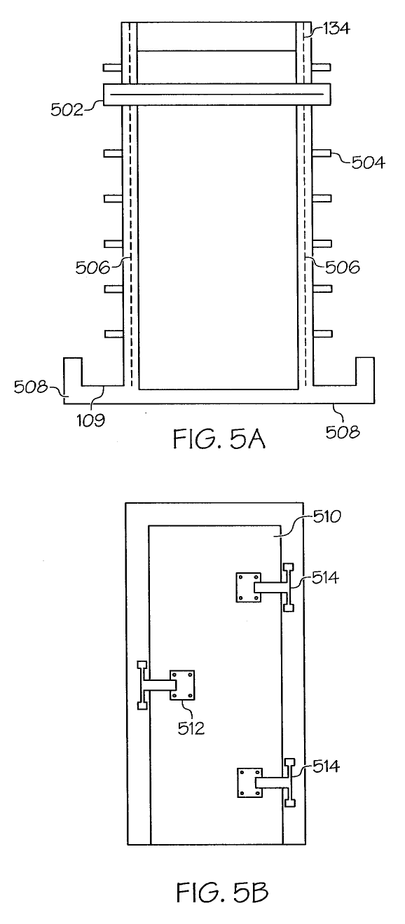

FIG. 1D depicts a diagram of an end view of the cryogenic stall 100 of FIG. 1D looking toward a second end wall 160a′, wherein the second end wall 106a′ comprises a door (describe further with respect to FIG. 5) in accordance with the principles of the present invention.

As depicted, the door may be integrated with the first end wall 106a. For example, the door may include a frame with an attached or integrated log. The log of the door may interlock with the lateral logs 107 such that the log of the door is stacked into second end wall 106a′ in vertical alignment with end logs 105. The door may comprise a swinging door that opens inwardly or outwardly about a hinge. Alternatively, the door may comprise a tambour door that raises. Furthermore, the frame of the door may comprise an interlock mechanism (described further with respect to FIGS. 2, 4 and 5) for attachment to the side of the lateral logs 107. For example, the interlock mechanism may include male rods extending outwardly from the door frame and female interlocks formed to receive the male rods. The female interlock may be integrated with the lateral logs 107. Other examples include any other attachment or stabilization mechanism, such as adhesives, hook and loop, magnets, etc.

In embodiments wherein the door frame does not include sufficient logs to engage with the notches 109 of all the lateral logs 107 beside the door at the first end wall 106a, stub logs (described with respect to FIG. 2) may be used to engage the corresponding notches 109 of the lateral logs 107 above and below the gaps.

Returning to FIG. 1A, a floor cover 111 may rest on the ground between each of the walls 104. Furthermore, the floor cover 111 may cover the entire area between walls 104. In some embodiments, the floor cover 111 is greater than the area between walls 104 and may rest on the inside of one or more walls 104. The floor cover 111 may be used to catch animal droppings. Therefore, a flexible material, such as a tarpaulin, may be used. Some embodiments include a material that can withstand the weight of a horse and the sharpness of horse hooves. Therefore, rubberized materials can also be used for the floor cover 111. In some embodiments, the floor cover 111 may be thermally conductive. Additionally, the floor cover 111 may be water resistant, such that the floor cover 111 is washable.

FIG. 2 depicts diagrams of individual logs 103, a vent plug 206, and a log plug 208 of the kit for building the cryogenic stall 100 of FIG. 1 in accordance with the principles of the present invention.

The logs 103 of the kit may include vent logs 110a and 110b, foundation logs 128a and 128b, and builder logs 130a and 130b.

The logs 103 of the kit may include logs 103 of various sizes. For example, a cross-section of the log 103 may be about 30 cm. The length of the log 103 may be between 30 cm and 365 cm, inclusive. For example, the length of the log 103 may be 30 cm, 60 cm, 120 cm, 180 cm, 240 cm, or 365 cm.

In some embodiments, the kit may include multiple logs 103 of similar and/or various sizes. The withers of a horse may typically stand about 180 cm from the ground. Therefore, kits for use with horses may comprise a sufficient number of logs 103 to build the walls 104 of the cryogenic stall 100 to at least a horse's withers. For example, the kit may contain fourteen respective logs 103 sized at each of the lengths exemplified above, such that each wall 104 of the chamber may be built by stacking each wall 104 seven logs 103 high. In other embodiments, the kit may include fourteen logs 103 of a first length and fourteen logs 103 of a second length. Thus, two respective lateral walls 107 may be built by stacking respective sets of seven logs 103 of the first length each (e.g. lateral logs 107). Furthermore, two respective end walls 106a, 106b may be built by stacking respective sets of seven logs 103 of the second length each (e.g. end logs 105).

However, embodiments also include sufficient logs 103 such that the walls 104 of the cryogenic stall 100 may be built above the withers of the horse. For example, the walls 104 may be built up to and including 366 cm. Thus, embodiments include kits having sufficient logs 103 to build the walls 104 between 183 cm and 366 cm.

End logs 105 may be 183 cm (6 feet) long. When the walls 104 are built, the interlocking of the end logs 105 with the lateral logs 107 may provide 122 cm (4 feet) of width between the inside of the lateral walls 108a, 108b.

The kit may also include one or more vent plugs 206. The vent plug 206 may be sized such that the vent plug 206 may be frictionally fit within vent 112. When vent plug 206 is within vent 112, the vent 112 may be at least partially block from transporting chilled air into cryogenic chamber 102. For example, vent plug 206 may comprise a central hole whereby the diameter of vent 112 is effectively reduced to the size of the central hole. Alternatively, vent plug 206 may comprise solid materials whereby the vent 112 is entirely blocked. Thus, the flow of chilled air into chamber 102 may be regulated by blocking and/or opening vents 112.

One or more log plugs 208 may also be included in the kit. These log plugs 208 may be shaped to frictionally fit with the notch 109 of the log 103. The log plugs 208 may be inserted into the notch 109 when the notch 109 is positioned such that the notch 109 does not engage another log 103.

One or more stub logs 202 may be included with the kit. The stub logs 202 may comprise one or two notches 109. The stub logs 202 may also be shorter than the end logs 105. For example, the stub logs 202 may be 30.5 cm (1 foot) long. In this manner, the stub logs 202 may be stacked vertically in interlocking manner with the notches 109 of lateral logs 107 such that the corresponding constructed end wall 106a, 106b provides sufficient space for a door, neck scarflet, or other structure.

The logs 103 may comprise a generally cylindrical body having a first end, a second end, and a length with rounded sides between the first end and second end. Alternatively, the logs 103 may have a prismatic structure, such as a hexagonal prism. Furthermore, the logs 103 may have a cross-section with at least one diameter.

The notches 109 may comprise a flat plane recessed into the surface of the log 103. The plane of the notch 109 may be parallel to a tangent plane of the surface of the log 103. The notch 109 may also comprise notch sides, which may be formed by the body of the log 103 that is not recessed. For example, the notch 109 may comprise a rectangular recess in which notch sides extend perpendicularly from the face of the notch 109 to the surface of the log 103. In some embodiments, the notch 109 may span 30.5 cm or less from the first notch side to the second notch side. Furthermore, the notches 109 of each of the logs 103 in the kit may be sized uniformly such that the respective notches 109 may fit within the notches 109 of the other logs 103 to form a perpendicular interlock with the other logs 103.

Furthermore, each notch 109 may be purposefully positioned on each log 103. For example, each log 103 may have two rounded sides opposite one another. Rounded can also include similar shapes, such as hexagonal, triangular, etc. The first notch 109a may be positioned on the first side 214 of the log 103 at 30.5 cm or less from the first end 210 of the log 103. The second notch 109b may be positioned on the second side 216 of the log 103 at the same corresponding distance from the first end 210 of the log 103. The third notch 109c may be positioned 30.5 cm or less from the second end 212 on the first side 214 of the log 103. Additionally, the fourth notch 109d may be positioned 30.5 cm or less from the second end 212 on the second side 216 of the log 103 at the same corresponding distance as notch 109c.

However, embodiments of the foundation logs 128a, 128b include the notches 109 on opposite sides and at similar ends having differing distances from the end of the log 103. For example, notch 109b may be positioned 30.5 cm or less from the first end of the log 128. Accordingly, notch 109a may be positioned the distance of notch 109b plus the span of notch 109b. In this manner, the notches of one side of the foundation log 128 may engage the log of the neck scarflet 132 or the door 134 at the first distance from the respective ends and the end walls 106a, 106b may be built upon the notches 109 at the second distance from the respective ends. In this embodiment, the end walls 106a, 106b may be constructed of alternating stub logs 202 and lateral logs 107. In some embodiments, the positioning of the notches 109 described above may be used with the builder logs 130a, 130b and/or the vent logs 110a, 110b.

Foundation logs 128a, 128b may include a flat side and a rounded (hexagonal prismatic, etc.) side opposite the flat side. The rounded side may comprise notches 109. For example, one notch 109 may be positioned within 30.5 cm of the first end of the foundation log 128 and another notches 109 may be positioned within 30.5 cm of the second end of the foundation log 128.

In some embodiments, the end foundations logs 128a may comprise a flat side. In other embodiments, the lateral foundation logs 128b may comprise a flat side. Embodiments include end foundation logs 128a and lateral foundation logs 128b having respective flat sides. However, the kit may include end foundation logs 128a and lateral foundation logs 128b lacking flat sides.

Embodiments also include rounded logs 103 having notches one the first side 214 and no notches on the second side 216. Thus, the opposite side may be rounded and lacking notches 109.

With regard to the description herein, the naming of the sides 214 and 216 is relative and arbitrary. Therefore, any description of the first side 214 can also apply to the second side 216. Furthermore, descriptions of the second side 216 can also be applied to the first side 214. Similarly, notches 109 are substantially similar and the letter designation a-d is given for clarity of the description. Any positioning described with respect to a specific label is given for clarity and may be applied as between other notches.

The logs 103 may comprise one or more female interlocks 204. The female interlocks 204 may be aligned down the length of the log 103 on one or more sides. The female interlock 204 may be shaped to receive a male interlock, such as a standalone rod or the male interlock of the neck scarflet frame or door frame. For example, the female interlock 204 may comprise a cylindrical hole in the body of the log 103. The female interlock 204 may extend into the body of the log 103 perpendicular to the tangent plane of the surface of the log 103 at the female interlock 204. Furthermore, the female interlock 204 may be parallel to the plane of the notch 109 of the log 103.

In some embodiments, the builder logs 130a, 130b may comprise respective female interlocks 204. The lateral builder log 130b may comprise the female interlock 204. The lateral foundation log 128b may comprise the female interlock 204. The lateral vent log 110b may comprise the female interlock 204. In other embodiments, the end logs 105 may comprise female interlocks 204.

The material of the logs 103 may comprise any material having sufficient compression strength to withstand the weight of the constructed wall 104 of the cryogenic stall 100. Example materials include metal, rigid plastics, rubber, etc. Furthermore, the material may comprise a thermal insulator such that the cryogenic chamber 102 may be cooled efficiently. Example materials include, rigid plastics, composites, other polymers, rubber, etc. Some logs 103 may also comprise a sealing material on the respective portions of the first side 214 and the second side 216 that meets another log 103 or the ground, when the stall 100 is constructed. Example materials for the sealing material include silicone, rubber, or any other material capable of forming a frictional seal when pressed against another surface.

Example kits include two end foundation logs 128a, two lateral foundation logs 128b, two end vent logs 110a, and two lateral vent logs 110b.

Additional kits include two end foundation logs 128a, two lateral foundation logs 128b, two end vent logs 110a, two lateral vent logs 110b, at least two end builder logs 130a, and at least two lateral builder logs. For example, between two and ten end builder logs 130a, inclusive, may be included and between two and ten lateral builder logs 130b, inclusive, may be included. In the examples disclosed, the end logs 107 (e.g. end builder logs 130a) may comprise stub logs 202.

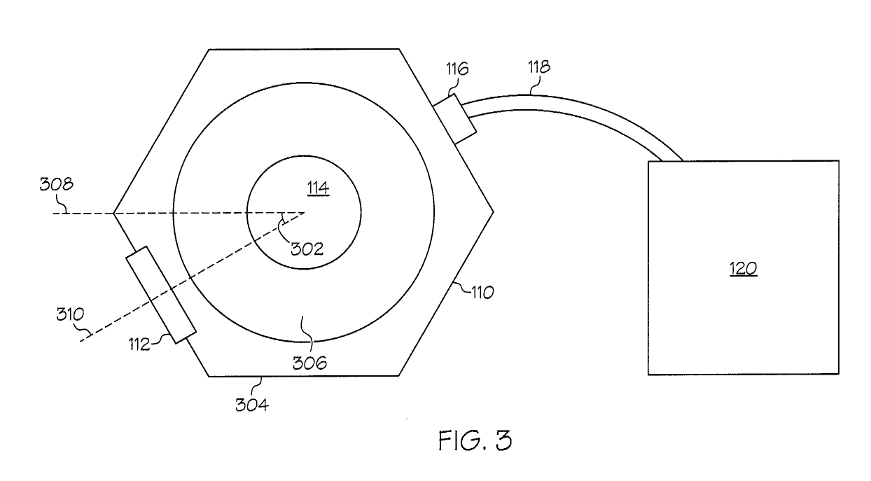

FIG. 3 depicts a cross-sectional view diagram of a vent log 110a, 110a′, 110b, 110b′ of FIG. 1 in accordance with the principles of the present invention.

Vent log 110a, 110a′, 110b, 110b′ may contain a vent 112 in fluid communication with a lumen 114. The lumen 114 may be in fluid communication with a connector 116. The connector 116 may be in fluid communication with a hose 118. The hose may be in fluid communication with the air chiller 120. Each of these structures may be in fluid communication with one another for the transport of chilled air from the air chiller 120 to the cryogenic chamber 102. However, omission of one or more of these parts is anticipated while retaining the same function. For example, the air chiller 120 may be mounted directly to the lumen 114, such that the connector 116 and the hose 118 are omitted and chilled air may be transported into chamber 102. Alternatively, embodiments include integrating the air chiller 120 with lumen 114.

The lumen 114 may span part or all of the length of the vent log 110. However, the lumen 114 may not necessarily penetrate either end of the vent log 110. The vent log 110a, 110a′, 110b, 110b′ may comprise one or more vents 112 that may be aimed into the chamber 102 when the stall 100 is constructed. Thus vents 112 may extend from the surface of the vent log 110a, 110a′, 110b, 110b′ to the lumen 114.

Similarly the connector 116 may comprise a structure that extends from the surface of the vent log 110a, 110a′, 110b, 110b′ to the lumen 114. The connector 116 may also extend away from the surface of the vent log 110a, 110a′, 110b, 110b′ such that the hose 118 may be connected to the connector 116. For example, the connection may be similar to the connection of a water hose, an air compressor, or any other connection sufficient for the transport of chilled air from the hose 118 through the connector 116. Connector 116 may also comprise an air filter.

Furthermore, the vent log 110a, 110a′, 110b, 110b′ may comprise an outer covering 304. The outer covering may comprise any material sufficient to retain shape and compression strength sufficient to support the vent log 110a, 110a′, 110b, 110b′ and any other logs 103 that may be placed on the vent log 110a, 110a′, 110b, 110b′ when the stall 100 is constructed. Within the outer covering 304, the vent log 110a, 110a′, 110b, 110b′ may comprise insulation 306. Insulation 306 may be the same material as outer covering 304 or different material than outer covering 304. Furthermore, insulation 306 may be formed around lumen 114. Additionally, insulation 306 may be contained within outer covering 304 or may be integrally formed with insulation 306. Insulation 306 may comprise any material sufficient for insulating chilled air for transport into the chamber 102. Example techniques and materials include fiberglass, a vacuum, certain plastics or composites, certain minerals, rubber, etc.

As described above, vent log 110a, 110a′, 110b, 110b′ may include notch 109. Furthermore, the vent log 110a, 110a′, 110b, 110b′ may include a parallel plane 308 that is parallel to the notch and bisects the vent log 110. Furthermore, vent log 110a, 110a′, 110b, 110b′ may contain vent plane 310 that bisects the vent 112 and intersects the parallel plane 308. Additionally, an offset angle 302 may comprise the angle between the parallel plane 308 and the vent plane 310.

In some embodiments, only the lateral vent log 110b contains a lumen 114 sufficient for carrying chilled air from the hose 118 to the interior of the chamber 102 (e.g. in fluid communication with the air chiller 120). In other embodiments, other lateral logs 107 may contain a lumen 114. However, embodiments also include one or more end logs 105 containing a lumen 114.

Because it may be desirable that the vents 112 of the vent log output chilled air onto the center of the back of the horse or other animal, the vents 112 may be directed toward the widthwise center of the stall 100 (e.g. about 60 cm away from each respective lateral wall 108) and 183 cm (6 feet) away from the ground. Therefore, an offset angle 302 of the vents 112 of the vent log 110a, 110a′, 110b, 110b′ may differ based on the intended height of the vent log 110a, 110a′, 110b, 110b′ for each respective kit.

For example, a kit having sufficient logs 103 to build the height of the walls to 183 cm may include vent logs 110a, 110b having vents 112 at an offset angle of about 0 degrees, wherein the offset angle 302 refers to the angle between the plane of the interlock notch 109 of the log shifted to the center of the vent log 110a, 110a′, 110b, 110b′ and the plane bisecting the vents when looking down the length of the vent log 110.

When the kit is configured for 183 cm wall height, e.g. sufficient logs 103 to build 183 cm high walls 104, the offset angle 302 of the vent log 110a, 110a′, 110b, 110b′ may be about 0 degrees.

When the kit is configured for 213 cm wall 104 height, the offset angle 302 of the vents 112 of the vent log 110a, 110a′, 110b, 110b′ may be about 26.6 degrees.

When the kit is configured for 244 cm wall 104 height, the offset angle 320 of the vents 112 of the vent log 110a, 110a′, 110b, 110b′ may be about 45 degrees.

When the kit is configured for 274 cm wall 104 height, the offset angle 302 of the vents 112 of the vent log 110a, 110a′, 110b, 110b′ may be about 56.3 degrees.

When the kit is configured for 305 cm wall 104 height, the offset angle 302 of the vents 112 of the vent log 110a, 110a′, 110b, 110b′ may be about 63.4 degrees.

When the kit is configured for 335 cm wall 104 height, the offset angle 302 of the vents 112 of the vent log 110a, 110a′, 110b, 110b′ may be about 68.2 degrees.

When the kit is configured for 366 cm wall 104 height, the offset angle 302 of the vents 112 of the vent log 110a, 110a′, 110b, 110b′ may be about 71.6 degrees.

The withers of a dog may stand from about 30 cm to about 89 cm. In embodiments wherein the kit is sized for a dog, the appropriate number of logs 103 may be included in the kit. For example, about two respective logs 103 of the first length and two respective logs 103 of the second length may be included in the kit. Alternatively, up to about six logs 103 having respective 30 cm widths may be included in the kit for each wall 104. The vents 112 of the vent logs 110a, 110b of these embodiments may be angled such that chilled air is aimed toward the widthwise center of the chamber 102 on the floor.

When the kit is configured for 61 cm wall height, the offset angle 302 of the vents 112 of the vent log 110a, 110a′, 110b, 110b′ may be about 45 degrees.

When the kit is configured for 91 cm wall height, the offset angle 302 of the vents 112 of the vent log 110a, 110a′, 110b, 110b′ may be about 56.3 degrees.

When the kit is configured for 122 cm wall height, the offset angle 302 of the vents 112 of the vent log 110a, 110a′, 110b, 110b′ may be about 63.4 degrees.

When the kit is configured for 152 cm wall height, the offset angle 302 of the vents 112 of the vent log 110a, 110a′, 110b, 110b′ may be about 68.2 degrees.

When the kit is configured for 183 cm wall height, the offset angle 302 of the vents 112 of the vent log 110a, 110a′, 110b, 110b′ may be about 71.6 degrees.

The logs 103 disclosed herein may be resized and the number of corresponding resized logs within the kit adjusted. For example, if the kit comprises logs 103 of 15 cm width (half of the width disclosed above), then double the number of logs 103 may be included to reach the appropriate height of the corresponding walls 104 (e.g. fourteen logs of half width in place of seven logs 103 of full width). The inverse is also true, if the kit comprises logs 103 of double width, half the number of logs 103 may be included to reach the appropriate height.

Each vent log 110a, 110a′, 110b, 110b′ may comprise a lumen 114 surrounded by an insulating material 306. The insulating material 306 may be further surrounded by an exterior housing 304. In some embodiments, the lumen 114 may span from about the proximal end to about the distal end of the log. However, the lumen 114 may not necessarily penetrate either end of the log 103. Instead, two orifices may be span from the lumen 114 through the insulating material 306 and through the exterior housing 304. In some embodiments, the insulating material 306 and the exterior housing 304 may be frictionally fit together. In other embodiments, the insulating material 306 and the exterior housing 304 may be integrally formed

The connector 116 may comprise any material sufficient for maintaining a connection with hose 118. Example materials include metal, plastic, rubber, etc. The vent 112 may comprise any material with sufficient rigidity to retain the shape of the vent 112. Example materials include metal, plastic, rubber, etc.

The notch 109 of the logs 103 may comprise a substantially horizontal plane that is recessed from the surface of the log 103. The offset angle may describe the angle between the horizontal plan of the notch 109 shifted to the center of the vent log 110a, 110a′, 110b, 110b′ and the plane bisecting the vent when viewing down the length of the vent log 110a, 110a′, 110b, 110b′ as in FIG. 1. The notch 109 may be recessed from the surface of the corresponding log 103 by one-quarter of the diameter of the log 103. In this manner, the logs 103 may be stacked in an alternating interlocking manner and may touch its vertical neighboring log 103. In some embodiments, the logs 103 may include notches 109 on opposite sides of the log 103 offset about 30 cm from each end of the log 103. In some embodiments, vent logs 110a, 110b and foundations logs 128 may include notches 109 on one side of the log 103.

The kit may optionally include log plugs 206. Each log plug 206 may be shaped to frictionally fit within the notch 109 of the log 103. Furthermore, the log plug 206 may be shaped such that the outer surface of the log plug 206 corresponds to the rounded outer surface of the log 103.

In some embodiments, the offset angle may be between about 0 and about 72 degrees. Embodiments also include an offset angle between about 45 degrees and about 72 degrees. Further embodiments include vents having an adjustable angle, wherein the angle may be set between about 0 degrees and about 72 degrees. Alternatively, the adjustable angle may be set between about 45 degrees and about 72 degrees. In order to facilitate adjustability, the vent may comprise a ball and socket vent, a rotating adjustable angle louver, a hinged vent, or any other structure that allows adjustability of the offset angle of the vent. The vent may comprise any material sufficient to retain shape and direct chilled air through the vent. Example materials include plastic, rubber, metal, etc.

Each log 103 may comprise a distal end and a proximal end. In some embodiments, the logs 103 may be of solid construction and the respective vent logs 110a, 110b for each corresponding wall may comprise a lumen 114 and two orifices. Connector section of logs may include notches 109, which comprise recessed, planes on the face of the logs 103. Each log 103 may contain four rectangular notches 109, each notch 109 spaced symmetrically along the length and symmetrically along the width of the log 103. A side of each rectangular notch 109 may be perpendicular to a lengthwise plane of the corresponding log 103. Each rectangular notch 109 may be similar in depth and size to the rectangular notches 109 of the other logs 103 in the kit. In this manner, respective logs 103 may be stacked perpendicularly at the notches 109, as depicted in FIGS. 1A-1D.

In some embodiments, the kit includes foundation logs 128a, 128b. Each foundation log 128 may comprise notches 109 that are symmetrically spaced along the length of one side of the log 128. Furthermore, the opposite side of the foundation log 128 may comprise a substantially planar section extending down the foundation log 128.

Each log 103 may comprise a surface sealing material that prevents airflow between vertically stacked logs 103. Example materials include rubber, silicone, etc.

The kits may also include vent plugs. In these embodiments, the flow of chilled air into the cryogenic chamber may be reduced by plugging one or more of the vents with a vent plug. Therefore, the vent may be shaped to receive the vent plug. Furthermore, the vent plug may correspond to the shape of the vent such that the vent plug may be frictionally fit into the vent. The vent plug may comprise any material sufficient for blocking airflow through the vents. Example materials include rubber, silicone, plastics, etc.

FIG. 4A depicts a diagram of an embodiment of the neck scarflet 132 of FIG. 1C in accordance with the principles of the present invention. The neck scarflet 132 may be included in the kit. For example, the log module kit may include a neck scarflet 132 comprising a frame, wherein the frame includes one or more vertical supports 402b and one or more horizontal supports 402a. The neck scarflet 132 may comprise attachment mechanisms 406 sufficient to attach a flexible material 408 to the frame. The neck scarflet 132 may include a flexible material 408 that may be generally rectangular. The flexible material 408 may comprise a neck hole 404. The flexible material 408 may be sufficiently flexible to shape to an animal's neck, when the animal's neck is through the neck hole 404. Example materials include leather, flexible plastics, and other polymers. The neck scarflet 132 may include a hole in the center of the rectangular material such that an animal's head may be extended through the hole. Sections of the flexible material 408 may also overlap, such that the flexible material 408 may be adjusted to the animal's neck. Furthermore, an attachment 410, such as hook and loop, magnets, adhesives, etc. may be positioned on the corresponding sections of the overlapping material 408 such that the overlapping material 408 may be secured around the animal's neck. In some embodiments, the animal's body may be within the chamber 102 and the animal's neck and head may extend outside the scarflet 132. In this manner, the flexible material 408 of the neck scarflet 132 may suppress the escape of evaporated gas from the cryogenic liquid within the chamber 102 such that the animal is not smothered. The flexible material 132 may be attached to a frame of the neck scarflet 132 by any attachment 406 sufficient to hold the neck scarflet 132 around the animal's neck. Example attachments 406 include brads, nails, hook and loop, weights, magnets, adhesives, integral formation, etc. The frame of the neck scarflet may comprise vertical supports and at least one horizontal support. The one or more horizontal supports may comprise one or more integrally formed logs 103 that extend beyond the neck scarflet 132 a sufficient distance such that on or more ends of each horizontal support may comprise notches 109 for interlocking with the lateral logs 103 of the perpendicular walls 104. Furthermore, the frame of the scarflet 132 may comprise hinges or a tambour mechanism for operating the frame of the scarflet 132 as a door. In this manner, an animal may be corralled into the chamber 102 through a door at the first end wall 106a. The animal may be backed out through the door at the first end wall 106a. Alternatively, the animal may be lead through the scarflet 132 having a door mechanism at the second end wall 106b. The frame of the scarflet 132 may also contain male interlocks extending orthogonally from the horizontal supports 402b and away from the flexible material 408.

FIG. 4B depicts an alternative neck scarflet 132 in accordance with the principles of the present invention. In this embodiment, the neck hole 404 may comprise a slit wherein flexible material 408 may be separated. Closures 410 may line corresponding sides of the neck hole 404. Furthermore, closures 410 may comprise magnets positioned to attract the corresponding magnet for keeping the neck hole 404 closed.

FIG. 4C depicts another alternative neck scarflet 132 wherein supports 402a, 402b may be attached to or integral with a door 416 in accordance with the principles of the present invention. Hooks 412 may extend from a face of the door 416 and may be used to tie back sections of flexible material 408. Furthermore, a bin 414 may extend from the outer surface of door 416. The bin 414 may be shaped to hold food or treats and may be positioned such that the animal may eat from bin 414 when the animal's neck is extended through neck hole 404. The bin 414 may also comprise a closable lid such that the animal can be prevented from eating from bin 414. The door 416 may rotate about hinges (not depicted) that are attaches to at least one wall 104. Alternatively, door 416 may be opened by the same mechanism as a tambour door. The bin 414 may be detachable and/or included with the kit independently of door 416. In some embodiments, door 416 may be 244 cm (eight feet) in height and 122 cm (four feet) in width.

FIG. 5A depicts a diagram of an embodiment of the door 132 of FIG. 1D in accordance with the principles of the present invention. The door may comprise a frame 506. The frame 506 may comprise hinges and support for a swinging door. Alternatively, the frame 506 may comprise a sliding mechanism for a tambour door cover 502. The frame 506 may also comprise a door frame log 508 that may include notches 109 for the engagement of other logs 103 in a similar manner as the neck scarflet 132. The frame 506 may also include male interlocks 504 extending orthogonally from the horizontal frame members. The male interlocks 504 may be sized and shaped to fit within the female interlocks 204 of the logs 103. A tambour door can include sliding flexible shutters or door pieces aligned with a track of the frame 506, wherein the shutters may be made of strips of rigid material. Furthermore, the strips of rigid material may be attached to a backing of canvas or other flexible material. In some embodiments, the track of the frame 506 may include an arcuate extension over the chamber 102.

FIG. 5B represents an alternative embodiment of a door 510 of the kit in accordance with the principles of the present invention. Door 510 may comprise hinges 514 that attach to the door 510 and the surrounding door frame. Hinges 514 may be configured to allow lateral rotation about an axis of rotation of the hinges 514. Furthermore, door 510 may comprise a latch 512 such that the door 510 can be secured in the closed position. However, embodiments of the door 510 exist wherein the hinges 514 are secured at the bottom of door 510 and the corresponding door frame. Thus, the axis of rotation may be at the bottom of the door 510. In this embodiment, the door 510 may open from the top and may be positioned as a ramp into the cryogenic chamber when fully open. In some embodiments, the length of latch 512 may span the width of door 510.

Returning to FIG. 3, the air chiller 120 may comprise cryogenic cooling system using a cryogenic liquid, such as liquid nitrogen, liquid carbon dioxide, liquid oxygen, or any other known cryogenic liquid or mixtures thereof. For example, the air chiller 120 may spray, release, mix, intermingle, etc. the cryogenic liquid into contained air of the air chiller 120. Alternatively, the cryogenic liquid may be stored with a partial pressure of a corresponding compressed gas. The compressed gas may then be decompressed into the air of the air chiller 120 to produce the chilled air. The resulting chilled air may be transferred through the hose 118, connector 116, lumen 114, and into the chamber 102. Thus, “chilled air” as referred to in this description refers to cryogenically cooled air. For example, the chilled air may be cooled sufficiently such that the cryogenic chamber 102 is cooled to −130 degrees Fahrenheit or cooler. Embodiments also include the chilled air cooling the cryogenic chamber 102 to −129 degrees Celsius (−200 degrees Fahrenheit) or cooler. Other embodiments include the chilled air of the air chiller 120 cooling the cryogenic chamber 102 to between −129 degrees Celsius (−200 degrees Fahrenheit) to −151 degrees Celsius (−240 degrees Fahrenheit).

Other structures and methods of operation for the air chiller 120 can include the air condition of AIR CONDITIONING APPARATUS USING LIQUID NITROGEN as described in U.S. Pat. No. 5,960,635, the entire contents of which are fully incorporated herein by reference. For example, this air conditioner can be used to produce chilled air within the air chiller 120. Additionally, hose 118 may comprise material sufficient to transport liquid nitrogen, cryogenically chilled air, decompressed nitrogen gas, etc. into cryogenic chamber 102. The solenoid control value 26 may be configured to allow cryogenic temperatures within the cryogenic chamber 120, as described above. Furthermore, housing 22 may correspond to a housing of the air chiller 120, and space 36 may correspond to air within the air chiller 120. Thus, the air within the chiller may be chilled and then transported out of the air chiller 120 via hose 118. The air chiller 120 may contain a connector formed with the housing and configured to connect with hose 118. The connector of the air chiller 120 may be similar in all respects to the connector 116. Fans 56 and 62 may direct the chilled air through the hose 118, when connected to the air chiller 120. Furthermore, housing 22 may comprise sufficient insulators to maintain the cryogenic temperatures of the air chiller 120. Example techniques and materials include plastics, fiberglass, certain minerals, double-layered materials surrounding a vacuum, etc.

The kit may also include a veterinary pulse oximeter, such as the SurgiVet V1030. Generally, pulse oximeters operate by measuring the transmittance of at least two wavelengths of light, typically red and infrared, through tissue containing blood. Some pulse oximeters emit red light at 660 nanometer wavelength and emit infrared light at 940 nm. The emitted light may be passed through human tissue and received by a photodiode of the pulse oximeter. At the wavelengths stated above, oxygenated hemoglobin absorbs more infrared light and allows more red light to pass through than deoxygenation hemoglobin. Thus, deoxygenated hemoglobin allows more infrared light to pass through and absorbs more red light. The veterinary pulse oximeter may be handheld or attached to the floor, floor cover 111, and/or a floor tray. For example, the veterinary pulse oximeter may be attached to the animal's tongue, hock, or hoof. The veterinary pulse oximeter may be positioned on the floor, floor cover 111, and/or a floor tray such that the animal may step into the pulse oximeter. Then, the animal's pulse and blood oxygen level may be detected. The pulse oximeter may be in electrical communication with the air chiller 120 such that the air chiller 120 prevents chilled air from entering hose 118 when the detected pulse or blood oxygen level is too low. In some embodiments, the veterinary pulse oximeter may alert an operator of the cryogenic chamber. In some embodiments, the pulse and blood oxygen may be displayed to an operator of the cryogenic chamber.

Furthermore, the kit may also include a humidity sensor, such as the Honeywell Humidlcon humidity and temperature detector models hlh 6000 Series, hlh 6100 Series, hlh7000 Series, hlh8000 Series, hlh9000 Series The Humidlcon sensors have respective humidity detection accuracies: ±4.5% relative humidity (RH) (HIH6000 Series), ±4.0% RH (HIH6100 Series), ±3.0% RH (HIH7000 Series), ±2.0% RH (HIH8000 Series), ±1.7% RH (HIH9000 Series). The humidity sensor may be place on or near floor cover 111 or a floor tray, such that the humidity sensor may detect increased humidity, such as that caused by evaporation from wet hooves. The humidity sensor may alert an operator not to begin cooling the cryogenic chamber 102 due to the risk of frostbite. The humidity sensor may be in electrical communication with the air chiller 102 such that air chiller 102 prevents chilled air from entering hose 118 when the detected humidity is too high.

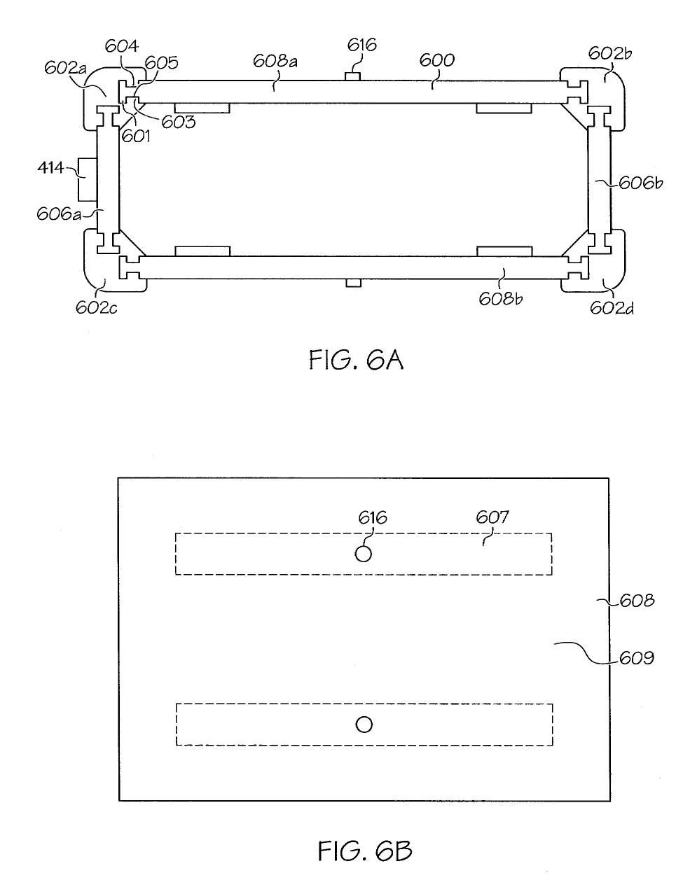

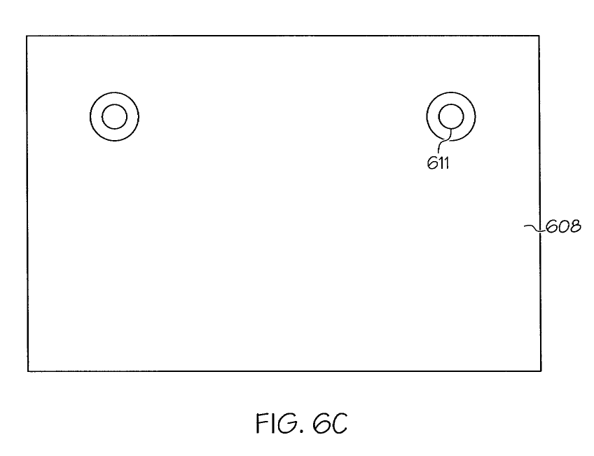

FIG. 6A depicts a representation of a cryogenic stall 600 in accordance with the principles of the present invention. Cryogenic stall 600 may be modular and may be similar in all respects to cryogenic stall 100. However, cryogenic stall 600 may comprise slab walls 606a, 606b and 608a, 608b rather than logs 103.

Lateral slab walls 608a, 608b may be 366 cm (twelve feet) in width whereas end slab walls 606a, 606b may be 122 cm (four feet) in width. Embodiments include both lateral slab walls 608a, 608b and slab walls 606a, 606b being 122 cm (four feet) in width. Embodiments include both lateral slab walls 608a, 608b and slab walls 606a, 606b being 183 cm (six feet) in width. First slab end walls 606a may comprise neck scarflet 132, which can include bin 414. Furthermore second slab end wall 606b may comprise a door similar in all respects to door 134. Lateral walls 608a, 608b may comprise connectors 616.

Cryogenic stall 600 may also include corners 602a, 602b, 602c, 602d. Each corner 602a, 602b, 602c, 602d may include two or more slots 604. The slots 604 may be positioned at a 90 degree angle relative to the opposite slot 604. Furthermore, each slot 604 may be configured to receive an end of a respective wall (e.g. end 601 of lateral slab wall 608a). In this manner, four corners 602a, 602b, 602c, 602d may be included with four slab walls 608a, 608b and 606a, 606b in order to construct cryogenic stall 600. Furthermore, slot 604 may include notch 603. Notch 603 may span part or all of the height of corner 602a, 602b, 602c, 602d. Each slab wall 606a. 606b and 608a, 608b may include groove 605, wherein groove 605 is configured to receive notch 603 such that the slab walls 606a, 606b and 608a, 608b are secured in the direction of the corresponding slot 604.

Slab walls 608a, 608b and 606a, 606b and corners 602a, 602b, 602c, 602d may be 244 cm (eight feet) in height or more. The slab walls 608a, 608b and corners 602a, 602b, 602c, 602d may comprise any material having sufficient rigidity to retain shape and may comprise insulating materials, as disclosed herein. The slab walls 608a, 608b and 606a, 606b may be referred to as “wall modules.” Furthermore, lateral slab walls 608a, 608b may be referred to as “lateral wall modules.” End slab walls 606a, 606b may be referred to as “end wall modules.” Slab walls 608a, 608b and 606a, 606b having vents may be referred to as “vent wall modules.”

FIG. 6B depicts a view of the outside of lateral slab wall 608a, 608b in accordance with the principles of the present invention. For example, the outside of lateral slab wall 608a, 608b may comprise connector 616. Connector 616 may be similar in all respects to connector 116. Connector 616 may extend to lumen 607 of lateral slab wall 608a, 608b. Lumen 607 may be similar in all respects to lumen 414. Lumen 607 may span part or all of the length of slab wall 608a, 608b.

FIG. 6C depicts a view of the inside of lateral slab wall 608a, 608b in accordance with the principles of the present invention. Vents 611 may extend through the inside surface of lateral slab wall 608a, 608b to the lumen 607. The angle of vents 611 may be determined relative to the angle between the plane bisecting the vents 611 and the plane of the inside surface of lateral slab wall 608a, 608b.

When the kit is configured for 61 cm vent height, the vent angle may be about 45 degrees.

When the kit is configured for 91 cm vent height, the vent angle may be about 33.7 degrees.

When the kit is configured for 122 cm vent height, the vent angle may be about 26.6 degrees.

When the kit is configured for 152 cm vent height, the vent angle may be about 21.8 degrees.

When the kit is configured for 183 cm vent height, the vent angle may be about 18.4 degrees.

When the kit is configured for 213 cm vent height, the vent angle may be about 15.9 degrees.

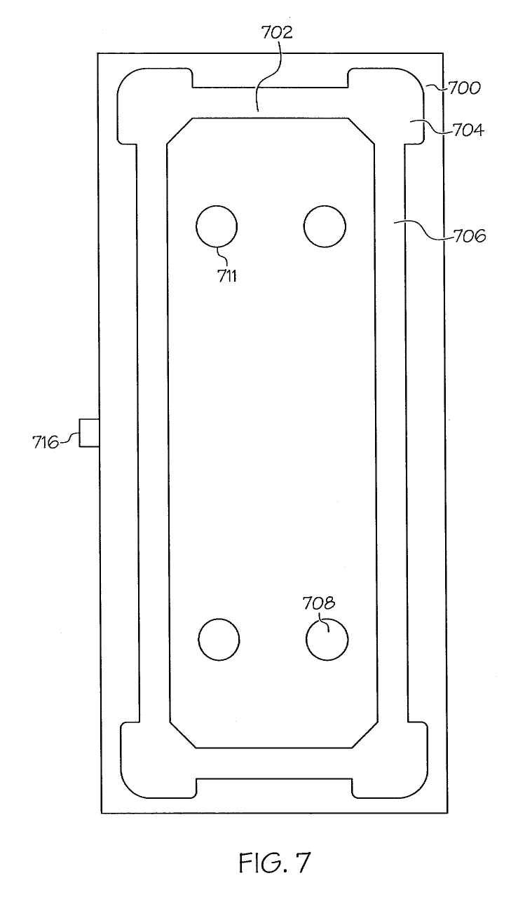

FIG. 7 represents a roof 700 of the kit in accordance with the principles of the present invention. The roof 700 may have a sufficient length and a sufficient width to cover the area within walls 606a, 606b and 608a, 608b, when the stall 600 is assembled. Therefore, the stall 600 may be at least 366 cm (12 feet) long and at least 122 cm (4 feet) wide.

Furthermore, the roof 700 may comprise one or more end wall groove 702 that correspond with the assembled position of the respective end walls 606a, 606b. Roof 700 may comprise one or more corner grooves 704 that correspond with the assembled position of the respective corners 602a, 602b, 602c, 602d. Roof 700 may comprise one or more lateral wall grooves 706 that may correspond with the assembled position of the respective lateral walls 608a, 608b. In this manner, the roof 706 may frictionally fit onto the top of the assembled walls 606a, 606b and 608a, 608b and corners 602a, 602b, 602c, 602d (e.g. the assembly depicted in FIG. 6).

The roof 700 may also comprise roof vents 706. The roof vents may be substantially similar to vents 611. Similar to vent 611, the angle of roof vents 706 may be adjustable. In some embodiments, the roof vents are perpendicular to a plane of the roof 700.

The roof 700 may also comprise connector 716. Connector 716 may be similar in all respects to connector 616. Furthermore, connector 716 may extend through the surface of roof 700 to a lumen (not depicted) of the roof 700. This lumen may be substantially similar to lumen 414. Furthermore, the roof lumen may extend between connector 716 and vents 708 such that the connector 716, roof lumen, and vents 708 are in fluid communication.

The roof 700 may comprise any material sufficiently rigid to retain the shape of the roof 700. Example materials include rigid plastics, fiberglass, rubber, certain polymers, etc. Furthermore, the roof 700 may contain sufficient thermal insulation around the roof lumen such that the chilled air remains chilled when passing through the vents 708. Example materials include fiberglass, certain minerals, rubber, etc.

In some embodiments, chilled air may be delivered through connector 716 from air chiller 120 through hose 118. Embodiments also include delivery of pharmaceuticals or other therapeutics. For example, antibiotics or antiparasitics may be applied.

FIG. 8 depicts a floor tray 800 of the kit in accordance with the principles of the present invention. The floor tray 800 may be substantially similar to the roof 700. For example, the floor tray 800 may comprise similar materials and may also comprise a corresponding lateral wall groove 806, corner groove 804, and end wall groove 802. Each of the respective grooves 806, 804, and 802 may correspond and frictionally fit with the assembled walls 606a, 606b and 608a, 608b and corners 602a, 602b, 602c, 602d.

Furthermore, floor tray 800 may comprise a sloping edge 808. The sloping edge may comprise a triangular prism having a flat edge with a height that is similar to the thickness of floor tray 800. The length of the sloping edge 808 may be similar to the width of the floor tray 800.

Floor tray 800 may also comprise heating coil 812. Heating coil 812 may comprise any material capable of resisting electricity to radiate heat. Examples could include one or more of copper, nickel, chromium, etc. Heating coil 812 may be contained within the material of floor tray 800 or on top of floor tray 800.

Floor tray 800 may comprise piping 810. Piping 810 may be contained within floor tray 800 or on top of floor tray 800. Piping 810 may be filled with water and connected to heating coil 812 such that the water may be heated. Thereafter, piping 810 may cycle the heated water and radiate heat. Furthermore piping 810 may in fluid communication with air chiller 120 such that the water may be chilled and the chilled water may be cycled through the piping 810. Thus, piping 810 can be used for the delivery of hot and/or cold therapy into the cryogenic stall 600.

Floor tray 800 may further comprise a scale positioned such that the weight of the animal may be determined by the scale. The scale may be in electrical communication with a computer. The scale may be a spring scale, piezoelectric scale, or any other scale capable of generating a weight measurement in electronic form. The scale may send the weight measurement to the computer. Embodiments of the present invention also include four scales in the four respective corners of the floor tray 800. The computer may receive the four respective weight measurements corresponding to the respective scales. Thus, weight shifting of the animal may be detected as the animal stands on the floor tray 800. For example, weight shifting may indicate laminitis in a horse.

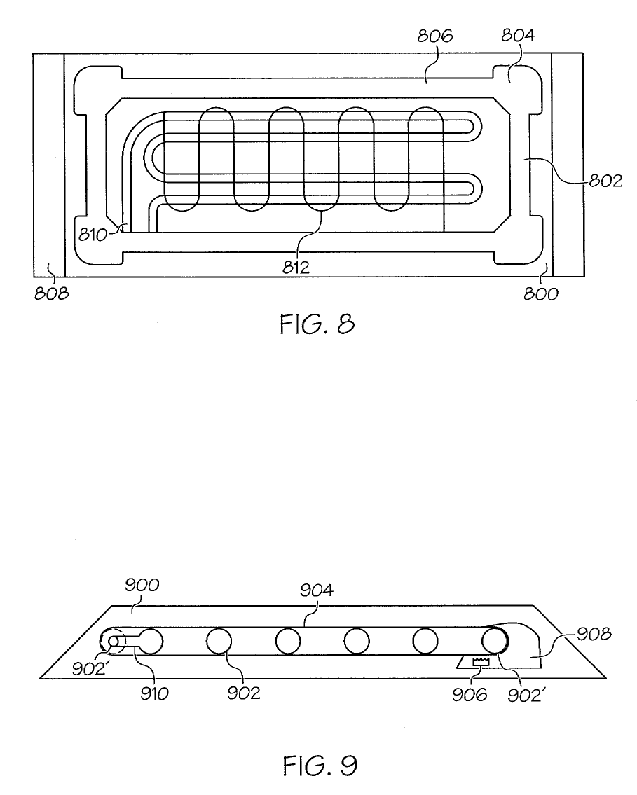

FIG. 9 represents a treadmill 900 of the kit in accordance with the principles of the present invention. In some embodiments, treadmill 900 may be integrally formed with, attached to, or placed upon floor tray 800. In other embodiments, treadmill 900 may be placed within any cryogenic stall disclosed herein.

Treadmill 900 may comprise two or more rollers 902. Furthermore, an end roller 902′ may be adjustable laterally such that the length of the treadmill 900 and the tension of belt 904 may be adjusted along a track 910. Belt 904 may be continuous. Belt 904 may be positioned to rotate around the rollers 902. Furthermore, the belt 904 may rotate such that any animal droppings left on the belt 904 may be transported to manure tray 908. Therefore, manure tray 908 may be positioned at an end roller 902′.

Brush 906 may be placed to brush the underside of belt 904. Thus, brush 906 may remove part or all of any manure from the belt 904. Brush 906 may be positioned over manure tray 908 such that removed manure may fall into the manure tray 908.

Belt 904 may comprise any material sufficient to withstand the tension of the rollers 902 and the hooves of horses. Example materials include rubber, certain polymers, polyurethanes, certain plastics, etc. Furthermore, the rollers 902 may be any size and shape for engaging the inside of belt 904 and moving belt 904 when the rollers 902 rotate in a uniform direction. Thus, the surface of the respective rollers may have a sufficient coefficient of friction to frictionally engage the belt 904. Example materials for the rollers include rubber, certain polymers, polyurethane, certain plastics, etc.

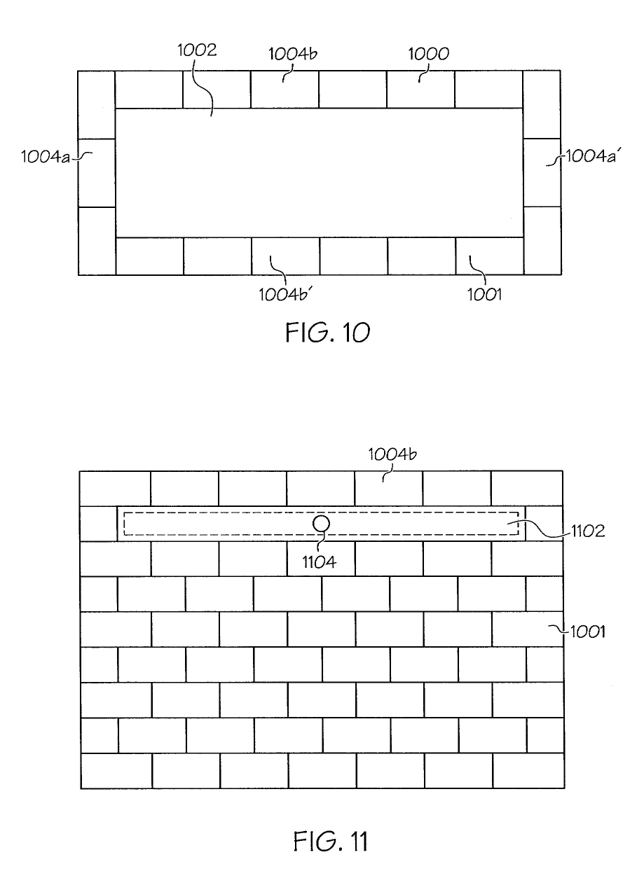

FIG. 10 depicts a cryogenic stall 1000 of the kit of the present invention. In some embodiments, cryogenic stall 1000 may comprise bricks 1001 as the building blocks for the walls 1004a, 1004a′, 1004b, 1004b′ of the cryogenic chamber. The bricks 1001 may be similar to the TOY BUILDING BRICK of U.S. Pat. No. 3,005,282, the contents of which are hereby incorporated by reference.

The bricks 1001 may be used to construct lateral walls 1004b, 1004b′ and end walls 1004a, 1004a′ of the cryogenic stall 1000. The walls 1004a, 1004a′, 1004b, 1004b′ may surround a chamber 1002. First end wall 1004a may comprise a neck scarflet 132. Furthermore, Second end wall 1004a′ may comprise a door. In some embodiments, the walls 1004a, 1004a′, 1004b, 1004b′ may be built to 274 cm (nine feet) in height. Furthermore, the walls 1004a, 1004a′, 1004b, 1004b′ may be built to the heights discloses for walls 110a, 110a′, 110b, 110b′ and/or walls 608a, 608b and 606a, 606b. (Walls 608a, 608b and 606a, 606b may be any height disclosed for walls 110).

FIG. 11 is a depiction of the inside of the first lateral wall 1004b of the cryogenic stall 1000 according to the principles of the present invention. The lateral walls 1004b, 1004b′ may comprise a vent brick 1102. The inside of the first lateral wall 1004b may comprise a vent 1104. Furthermore, the bricks 1001 may be fit together to construct the lateral wall 1004b. Walls 1004a, 1004a′, 1004b, 1004b′ constructed of bricks 1001 may be referred to as “wall modules.”

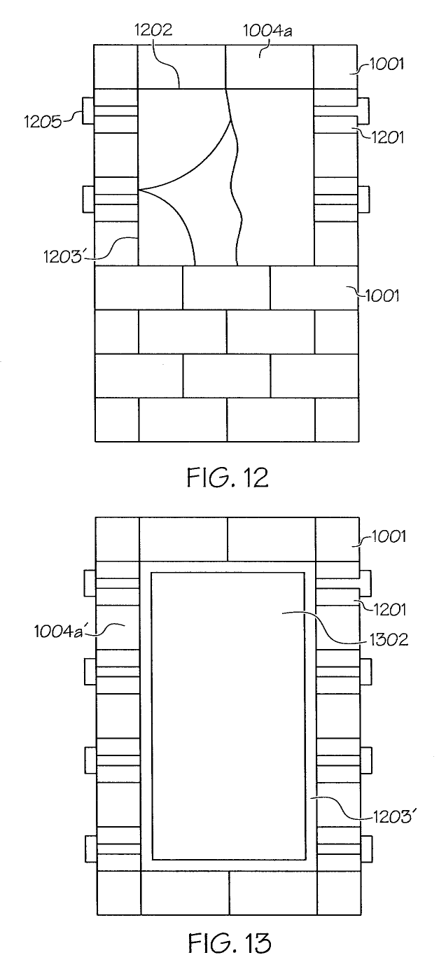

FIG. 12 is a depiction of the outside of the first end wall 1004a of the cryogenic stall 1000 according to the principles of the present invention. The first end wall 1004a may comprise neck scarflet 1202. Neck scarflet 1202 may be similar in all respects to neck scarflet 132. A frame 1203′ of the neck scarflet 1202 may anchor the neck scarflet 1202 into the first end wall 1004a. Furthermore, ends walls 1004a, 1004a′ may comprise end bricks 1201. End bricks 1201 may be similar in all respects to bricks 1001. However, end bricks 1201 may be half the length of bricks 1001. Furthermore, end bricks 1201 may be anchored to frame 1203′ via male interlocks 1205 may extend through some of the end bricks 1201 and into frame 1203′. As can be seen in all depictions of the walls 1004a, 1004a′, 1004b, 1004b′ of the cryogenic stall 1000, the bricks 1001 may be built in an alternating offset pattern such that the majority of the ends of the bricks 1001 do not align. Thereby, breaks in the walls 1004a, 1004a′, 1004b, 1004b′ may be prevented. However, the alternating overlap may cause gaps that are about the size of one half the length of the bricks 1001. In some embodiments, the width of the bricks 1001 may equal half the length of the bricks 1001. Therefore, a brick 1001 may be placed perpendicularly in the gap and may serve to anchor the walls 1004a, 1004a′, 1004b, 1004b′ together at the corners. However, some sections of the end walls 1004a, 1004a′ may require space for the neck scarflet 1202 or the door. Therefore, a perpendicular brick 1001 may obstruct the neck scarflet 1202 or the door. In these embodiments, the end brick 2101 may be placed in the gap.

FIG. 13 depicts the second end wall 1004a′ of the cryogenic stall 1000 according to the principles of the present invention. Second end wall 1004a′ may comprise door 1302. Door 1302 may be similar in all respects to door 134 and/or door 510. As can be seen, alternating bricks 1001 and end bricks 1201 may be built around the door 1302.

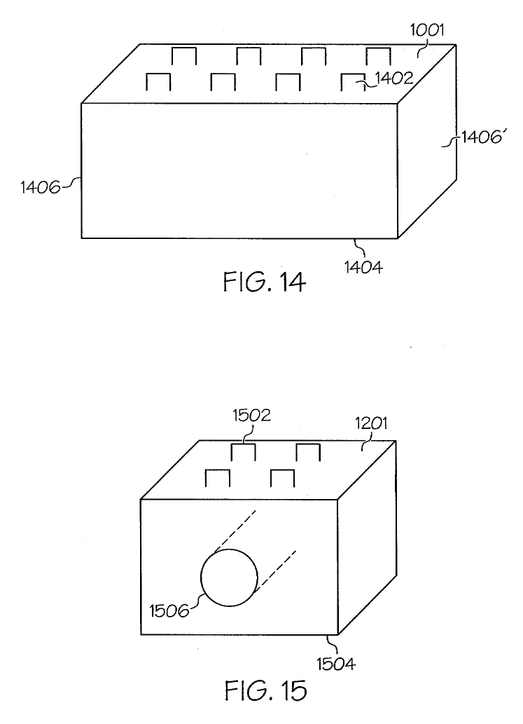

FIG. 14 depicts brick 1001 of the kit for the cryogenic stall 1000 in accordance with the principles of the present invention. The brick 1001 may have a length, a width, and a height. In some embodiments, the length may be about 61 cm (two feet). The width may be about 30.5 cm (one foot). The height may be about 30.5 cm (one foot).

The brick 1001 may have an end 1406. The end 1406 may comprise surface materials sufficient to create a seal when stacked against another end of another brick. For example, the surface materials may comprise rubber, low-density plastics, silicone, adhesives, certain polymers, etc. Furthermore, these surface materials may also be present on the surface of the top of the brick 1001 and the male connector 1402 and/or the bottom and the female connector 1404 such that a seal may be created when bricks 1001 are stacked upon one another.

Brick 1001 may also comprise a male connector 1402. In some embodiments, the male connector 1402 comprises circular orthogonal projections extending from the top surface of the brick 1001. Brick 1001 may comprise female connector 1404. Female connector may comprise any frictional fit receptacle for receiving male connector 1402. For example, female connector 1404 may comprise an open area with circular projections configured to engage the circular projections of the male connector 1402. In this manner, the female connector 1404 projection may have a diameter approximately equal to the diagonal distance between two male connector 1402 projections. Furthermore, the projection of the female connector 1404 may be positioned to frictionally fit between four of the projections of the male connector 1402. In this manner, walls 1004a, 1004a′, 1004b, 1004b′ may be constructed by the frictional engagement of corresponding male connectors 1402 and female connectors 1404 of the bricks 1001.

Alternatively, female connectors 1404 may comprise corresponding recesses configured to receive male connectors 1402 such that female connectors 1404 are frictionally fit to male connectors 1402.

FIG. 15 depicts an end brick 1201 of the kit for constructing the cryogenic stall 1000 in accordance with the principles of the present invention. End brick 1201 may comprise male connectors 1502 that may be similar in all respects to male connectors 1402. Furthermore, end brick 1201 may comprise female connectors 1504 that may be similar in all respects to female connectors 1404. Therefore, end brick 1201 can frictionally engage with bricks 1001 as well as other end bricks 1201 in the same manner as bricks 1001 engage other bricks 1001.

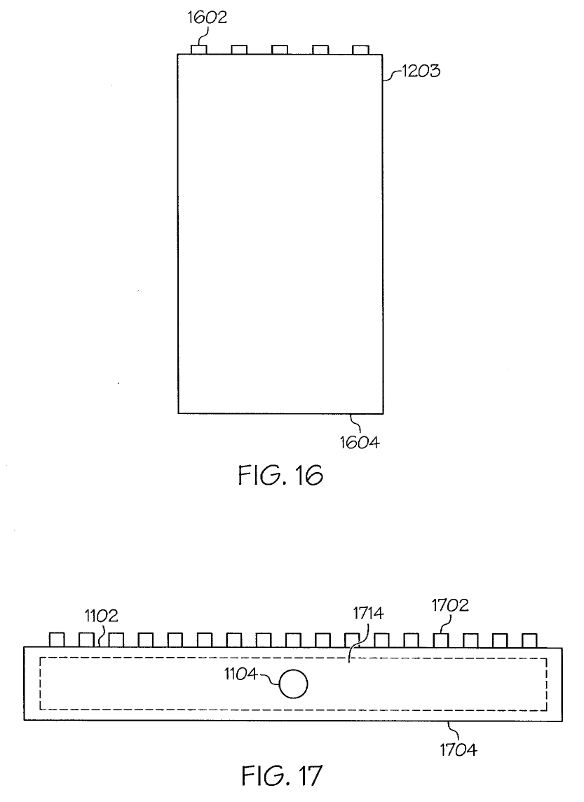

FIG. 16 depicts a frame 1203 of the kit in accordance with the principles of the present invention. The frame 1203 may be sized to contain a neck scarflet 132, a door 1302, or a window. The frame 1203 may be attached to and/or integrally formed with the neck scarflet 132, the door 1302, or the window. Furthermore, the frame 1203 may comprise male connectors 1602 and female connectors 1604. The male connectors 1602 may be similar in all respects to male connectors 1402 and/or 1502. The female connectors 1604 may be similar in all respects to female connectors 1602 and/or 1604.

FIG. 17 depicts a vent brick 1102 of the kit in accordance with the principles of the present invention. The vent brick 1102 may comprise a vent 1104. In some embodiments, the vent 1104 may be similar in all respects to vent 611. Furthermore, the vent brick 1102 may comprise a lumen 1714. The lumen 1714 may be similar to lumen 607 in all respects. The vent brick 1102 may comprise a connector (not depicted in FIG. 17) that is similar to connector 116 in all respects. Therefore, the vent 1104 may be in fluid communication with the connector via the lumen 1714.

The vent brick 1102 may comprise male connectors 1702. In some embodiments, the male connectors 1702 may be similar in all respects to male connectors 1402 and/or 1502. The vent brick 1102 may comprise female connectors 1704. The female connectors 1704 may be similar in all respects to female connectors 1404 and/or 1504.

The vent 1104 may be adjustable in offset angle. Alternatively, the offset angle of vent 1104 may follow the pattern for the angle of vent 611 dependent of the height of vent 1104 relative to the ground. The vent brick 1102 may be 61 cm (two feet) long, 122 cm (4 feet) long, 183 cm (6 feet) long, 244 cm (8 feet) long, 305 cm (10 feet) long, and/or 366 cm (12 feet) long. The vent brick 1102 may be 30.5 cm (one foot) wide and 30.5 cm (one foot) in height.

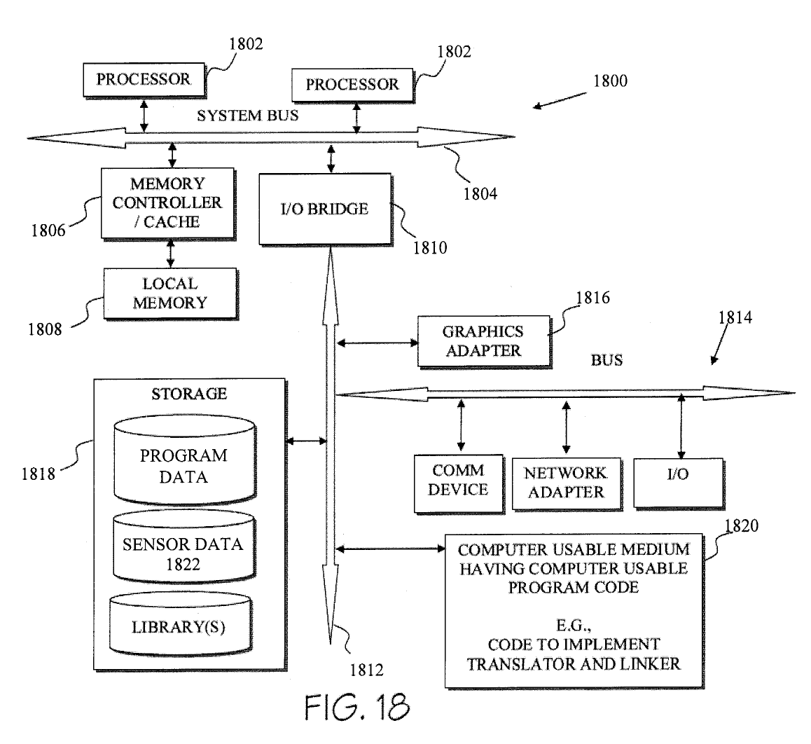

FIG. 18 illustrates a block-level diagram of a computer 1800 in accordance with the principles of the present invention. The computer 1800 may have a computer readable storage medium for implementing functions comprising aspects of the method detailed above. Computer 1800 may comprise a symmetric multiprocessor (SMP) system or other configuration including a plurality of processors 1802 connected to system bus 1804. Alternatively, a single processor 1802 may be employed. Also connected to system bus 1804 is memory controller/cache 1806, which provides an interface to local memory 1808. An I/O bridge 1810 is connected to the system bus 1804 and provides an interface to an I/O bus 1812. The I/O bus 1812 may be utilized to support one or more buses and corresponding devices, such as bus bridges 1814, input output devices (I/O devices), storage, network adapters, etc. Thus, a network adapter may be coupled to the system to enable the data processing system to become coupled to other data processing systems or remote printers or storage devices through intervening private or public networks.

Also connected to the I/O bus 1812 may be devices such as a graphics adapter 1816, storage 1818 and a computer usable storage medium 1820 having computer usable program code embodied thereon. The computer usable program code may be executed, e.g., by the processor(s) to implement any aspect of the present invention, for example, to receive, process, and/or store sensor data 1822. In some embodiments, sensor data 1822 can include one or more of: one or more weight measurements received from the scale(s), a pulse measurement received from the pulse oximeter, a blood-oxygen measurement received from the pulse oximeter, a humidity measurement received from the humidity sensor, and/or a temperature reading from a thermometer within the cryogenic chamber. Moreover, the computer usable program code may be implemented in the local memory 1808 or other suitable storage medium.

The storage 1818 may store resources useful in implementing the features previously described. For instance, the storage 1818 can store the computer instructions which, when executed, implement the functions of receiving and manipulating the sensor data 1822. Furthermore, the storage 1818 may comprise program data for communicating with the air chiller 120 or any other electrical component disclosed herein, as well as any useful libraries for manipulating the storage of sensor data 1822 and/or program data.

The computer 1800 may be in electrical communication with one or more of: the scale, the pulse oximeter, the humidity sensor, the thermometer, and/or the air chiller 120. Furthermore, the computer 1800 may store thresholds for each value. For example, when a temperature, humidity, pulse, blood-oxygen, time, etc. beyond a predetermined threshold is received, the computer 1800 may send a poweroff signal to the air chiller 120. The computer 1800 may store a lower threshold for temperature, an upper threshold for humidity, an upper threshold and a lower threshold for pulse, an upper and a lower threshold for blood-oxygen, an upper threshold for time, etc. Each of these predetermined thresholds may be determined by the end user. Additionally, the computer 1800 may add and/or compare weight measurements of four respective scales at corresponding corners of the floor tray 800. For example, if the animal continually shifts weight off of one leg, the corresponding scale may detect a lighter weight whereas the remaining scales may detect a heavier weight. Furthermore, if the animal favors one leg, the animal may not put much of its weight on the favored leg. Therefore, the computer may indicate problems, such as laminitis, when the measured weight at one scale is less than the measured weights at the remaining scales. Measurements indicating repeated weight shifting may indicate problems, such as laminitis in horses. For example, laminitis may be indicated by the computer 1800 when persistent weight shifting of over 7 percent of a horse's weight occurs.

The previous description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with each claim's language, wherein reference to an element in the singular is not intended to mean “one and only one” unless specifically so stated, but rather “one or more.” All structural and functional equivalents to the elements of the various embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.

What is claimed is:

1. A kit for the assembly of a cryogenic chamber, comprising:
at least four wall modules comprising respective bodies of insulation;
wherein the at least four wall modules comprise:
at least two end wall modules and at least two lateral wall modules,
wherein at least one of end wall modules or lateral wall modules comprises a vent wall module comprising:
a lumen surrounded by insulation,
a connector extending from a surface of the vent wall module to the lumen, and
a vent extending from the surface of the vent wall module to the lumen,
wherein the connector is in fluid communication with the vent via the lumen; and
an air chiller having a first interface shaped to operatively engage the connector of the vent module, wherein the air chiller is in fluid communication with the vent when the first interface and the connector are operatively engaged.

2. The kit of claim 1, further comprising a neck scarflet,
wherein the neck scarflet comprises:
a flexible material extending from a neck hole to a frame, and
the frame configured to engage at least one of the at least four wall modules by integral formation, log notches, or a brick connector.

3. The kit of claim 1, further comprising a door,
wherein the door comprises a hinged closing mechanism or a tambour closing mechanism, and
a frame of the door is configured to engage at least one of the at least four wall modules by integral formation, log notches, or a brick connector.

4. The kit of claim 1, wherein the at least four wall modules comprise log modules;
each log module comprising:
a first side comprising a first notch at a first end and a second notch at a second end; and
a second side opposite the first side, the second side comprising a third notch at the first end and a fourth notch at the second end.

5. The kit of claim 1, wherein the at least four wall modules comprise slab modules;
the slab modules comprising:
at least one lateral slab module that is at least 183 cm (six feet) in height and at least 244 cm (eight feet) in length;
at least one end slab module that is at least 183 cm (six feet) in height and at least 122 cm (four feet) in length;
wherein the at least one vent module comprises at least one vent slab module.

6. The kit of claim 5, further comprising:
at least one corner, the corner comprising a slot configured to receive an end of the slab wall module,
wherein the corner is at least 183 cm (six feet) in height.

7. The kit of claim 6, wherein the slot of the corner comprises a notch configured to engage a groove of the end of the slab wall module.

8. The kit of claim 7, wherein the end of the slab wall module comprises the groove configured to engage the notch of the corner.

9. The kit of claim 5, further comprising:
a floor tray comprising
at least one end wall groove configured to receive a portion of the at least one end wall;
at least one lateral wall groove configured to receive a portion of the at least one lateral wall;
wherein the at least one lateral wall groove is orthogonal to the at least one end wall groove.

10. The kit of claim 9, the floor further comprising a heating element and a piping.

11. The kit of claim 5, further comprising:
a roof comprising
at least one end wall groove configured to receive a portion of the at least one end wall;
at least one lateral wall groove configured to receive a portion of the at least one lateral wall;
wherein the at least one lateral wall is orthogonal to the at least one end wall.

12. The kit of claim 11, the roof further comprising
a connector extending from a surface of the roof to a lumen;
at least one vent extending from an interior surface of the roof to the lumen such that the vent and the connector are in fluid communication via the lumen;
wherein the interior surface of the roof is the area bounded by the at least one end wall groove and the at least one lateral wall groove.

13. The kit of claim 1, wherein the at least four wall modules comprise sufficient brick modules to build at least one lateral wall that is at least 183 cm (six feet) in height and at least 244 cm (eight feet) in length and at least one end wall that is at least 183 cm (six feet) in height and at least 122 cm (four feet) in length.

14. A vent wall module for a cryogenic chamber, comprising:
a lumen surrounded by an insulation,
a vent extending from a surface of the vent wall module to the lumen, and
a connector extending from the surface of the vent wall module to the lumen,
wherein the connector and vent are in fluid communication via the lumen.

15. The vent wall module of claim 14,
wherein the vent wall module comprises a vent log;
the vent log comprising:
a first side comprising a first notch at a first end and a second notch at a second end; and
a second side opposite the first side, the second side comprising a third notch at the first end and a fourth notch at the second end,
a vent extending from a surface of the vent log to the lumen, and
a connector extending from the surface of the vent log to the lumen such that the connector is in fluid communication with the vent via the lumen,
an offset angle of the vent, wherein the offset angle is determined by the angle of an intersection of a plane bisecting the vent and a parallel plane of the notch bisecting the vent log lengthwise.

16. The vent wall module of claim 15, wherein the offset angle is between 26 degrees and 72 degrees, inclusive.

17. The vent wall module of claim 15, wherein the offset angle is between 45 degrees and 72 degrees, inclusive.

18. The vent wall module of claim 14,
wherein the vent wall module comprises a vent slab wall;
the vent slab wall comprising:
a height of at least 183 cm (six feet),
a length of at least 122 cm (four feet),
a lumen in the vent slab wall,
a connector extending from a surface of the vent slab wall to the lumen,
a vent extending from the surface of the vent slab wall to the lumen such that the vent and the connector are in fluid communication via the lumen, and
an offset angle of the vent determined by an intersection of a plane bisecting the vent and a plane of the surface through which the vent extends.

19. The vent wall module of claim 18, wherein the offset angle is between 16 degrees and 45 degrees, inclusive.

20. The vent wall module of claim 14,
wherein the vent wall module comprises a vent brick module;
the vent brick module comprising:
a male connector on the top surface of the vent brick,
a female connector on the bottom surface of the vent brick,
the vent extending from a surface of the vent brick to the lumen,
the connector extending from the surface of the bent brick to the lumen such that the vent and the connector are in fluid communication via the lumen, and
an offset angle of the vent determined by an intersection of a plane bisecting the vent and a plane of the surface through which the vent extends.

21. The vent wall module of claim 20, wherein the offset angle is between 16 degrees and 45 degrees, inclusive.
